(19) United States
(12) Patent Application Publication
(10) Pub. No.: US 2012/0046633 A1
(43) Pub. Date: Feb. 23, 2012

(54) URINE ABSORBENT POUCH FOR MALE INCONTINENCE

(76) Inventors: Miyuki Okawa, Mima-gun (JP); Akiko Tatsukawa, Mima-gun (JP); Hirofumi Miyake, Mima-gun (JP)

(21) Appl. No.: 13/266,639

(22) PCT Filed: May 11, 2010
(86) PCT No.: PCT/JP2010/003195

§ 371 Date: Oct. 27, 2011

(30) Foreign Application Priority Data
May 22, 2009 (JP) 2009-123783

(51) Int. Cl. A61F 13/45

(52) U.S. Cl. 604/385.24

(57) ABSTRACT

The absorbent product for male comprises a bag-like main body part having an opening at an upper end, and two hydrophilic internal sheets which are band-like members located in the main body part. An upper end part of each internal sheet is fixed on an upper part of the main body part and the other portions are not bonded to the main body part. Internal sheet elastic members each extending in an up-down direction are bonded to each internal sheet, and by contraction of these members, gathers are formed in the internal sheet. In the absorbent product, since urine moves in the internal sheet comparatively slowly through the gathers formed in the internal sheet, a time required for the urine to reach the bottom part of the bag part can be increased. As the result, a large area of absorbent part can be utilized to absorb the urine effectively.

### TECHNICAL FIELD

The present invention relates to an absorbent product for receiving urine from a male wearer.

### BACKGROUND ART

A bag-like absorbent product in which the male genitalia of a wearer is housed, is conventionally used as one of absorbent products for receiving excrement from a wearer. For example, a urine absorbent appliance for male disclosed in Japanese Patent Application Laid-Open No. 2004-159724 (Document 1) comprises a bag-like body where an absorber is provided between a water-impervious film and a water-pervious film and a housing opening part formed around an opening of the bag-like body, and the housing opening part can expand and contract to hold the root of the male genitalia (penis and testis) of a wearer which is housed in the bag-like body.

In the urine absorbent appliance for male like Document 1, since excreted urine promptly reaches the bottom part of the bag-like body, only a portion in the vicinity of the bottom part of the bag-like body is used for absorption of urine. Thus, there may be a case where whole urine can not be absorbed and a certain amount of urine which is not absorbed by the absorber flows back with postural change of the wearer to leak out through a gap between the opening and the male genitalia.

### SUMMARY OF INVENTION

The present invention is intended for an absorbent product for receiving urine from a male wearer. It is an object of the present invention to utilize a large area of an absorbent part to absorb urine effectively.

The absorbent product according to the present invention comprises: a bag part which is formed of a water-repellent or liquid-impervious sheet member and which has an opening at an upper end into which the male genitalia of a wearer is inserted; a liquid-pervious internal sheet located in the bag part; an absorbent part for absorbing urine from the wearer, the absorbent part being located between the bag part and the internal sheet; and an elastic member which is bonded to the internal sheet, gathers being formed in at least part of the internal sheet by contraction of the elastic member.

In the present invention, since a time required for urine to reach the bottom part of the bag part is increased by the gathers, a large area of the absorbent part can be utilized to absorb urine effectively.

Preferably, the gathers are formed at a portion of the internal sheet opposed to a lower part of the bag part, and more preferably, the gathers are formed from an upper part to a lower part of the internal sheet.

According to a preferred embodiment of the present invention, the absorbent product further comprises a liquid-pervious top sheet which is located between the absorbent part and the internal sheet and which covers an inner surface of the bag part, wherein the internal sheet is a band-like member whose upper end part is fixed on an upper part of the bag part. It is therefore possible to prevent the absorbent product from becoming bulky.

In this case, the absorbent product further comprises an internal sheet absorbent part which is attached to the internal sheet. As the result, absorbency can be increased.

According to an aspect of the present invention, the internal sheet absorbent part comprises a plurality of absorbent elements which extend in an up-down direction and which are arranged in a direction orthogonal to the up-down direction, and the elastic member is a string-like member or a band-like member extending in the up-down direction and the elastic member is bonded to the internal sheet at an area between the plurality of absorbent elements.

According to another aspect of the present invention, a nonexistence area of absorber lying across the internal sheet absorbent part in a direction orthogonal to the up-down direction is provided on the internal sheet absorbent part.

These and other objects, features, aspects and advantages of the present invention will become more apparent from the following detailed description of the present invention when taken in conjunction with the accompanying drawings.

### BRIEF DESCRIPTION OF DRAWINGS

FIG. 1 is a front view of an absorbent product.

FIG. 2 is a cross-sectional view of the absorbent product.

FIG. 3 is a developed view of the absorbent product.

FIG. 4 is a cross-sectional view of an internal sheet.

FIG. 5 is a view showing another example of internal sheet absorbent part.

FIG. 6 is a view showing still another example of internal sheet absorbent part.

FIG. 7 is a view showing still another example of internal sheet absorbent part.

### DESCRIPTION OF EMBODIMENTS

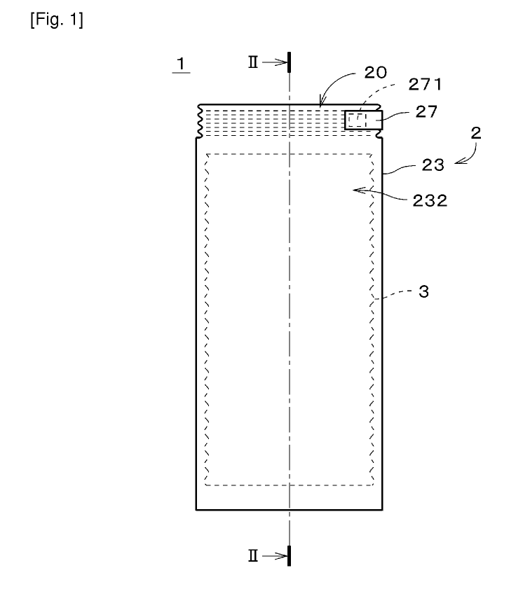

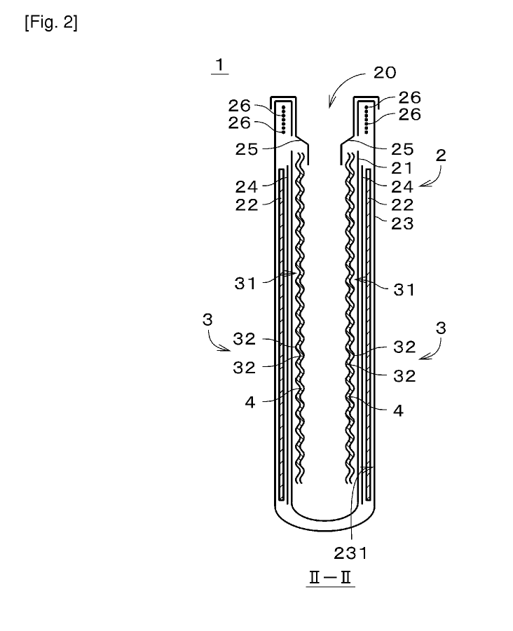

FIG. 1 is a front view of an absorbent product 1 in accordance with a preferred embodiment of the present invention. FIG. 2 is a cross-sectional view of the absorbent product 1 at the position indicated by arrows II-II in FIG. 1. The absorbent product 1 is for male and it is worn on the male genitalia of a wearer to receive (catch) urine from the wearer.

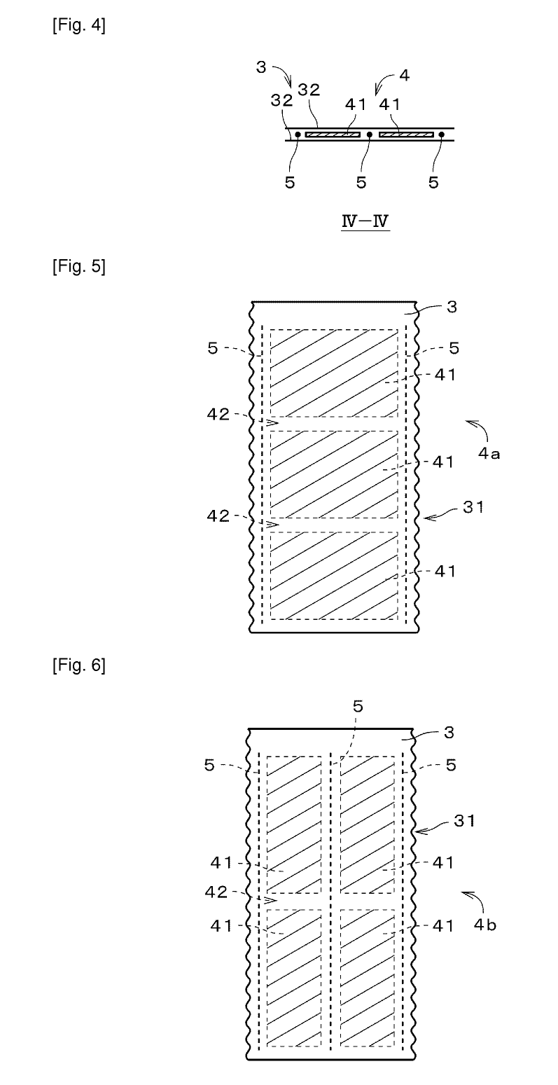

As shown in FIGS. 1 and 2, the absorbent product 1 comprises a bag-like main body part 2 having an opening 20 at an upper end which is one end in an up-down direction (it corresponds to a top and bottom direction in FIGS. 1 and 2 and it is not limited to a direction of gravitational force in the state where the absorbent product 1 is worn by the wearer) and two internal sheets 3 which are band-like members located in the main body part 2. The internal sheets 3 are liquid-pervious sheet members whose upper end parts are fixed on an upper part of the main body part 2 and in the preferred embodiment, they are hydrophilic sheet members. As shown in FIG. 2, the two internal sheets 3 face each other in the main body part 2 and portions of each internal sheet 3 excluding the upper end part are not bonded to the main body part 2. An internal sheet absorbent part 4 for absorbing urine from the wearer is attached to each internal sheet 3. In FIG. 2, hatching lines are drawn at the internal sheet absorbent part 4 for easy understanding of the drawing (the same applies to after-mentioned absorbent parts 22 of the main body part 2). In addition, respective constituents of the absorbent product 1 are drawn to be slightly apart from one another for easy understanding of the drawing (the same applies to FIG. 4).

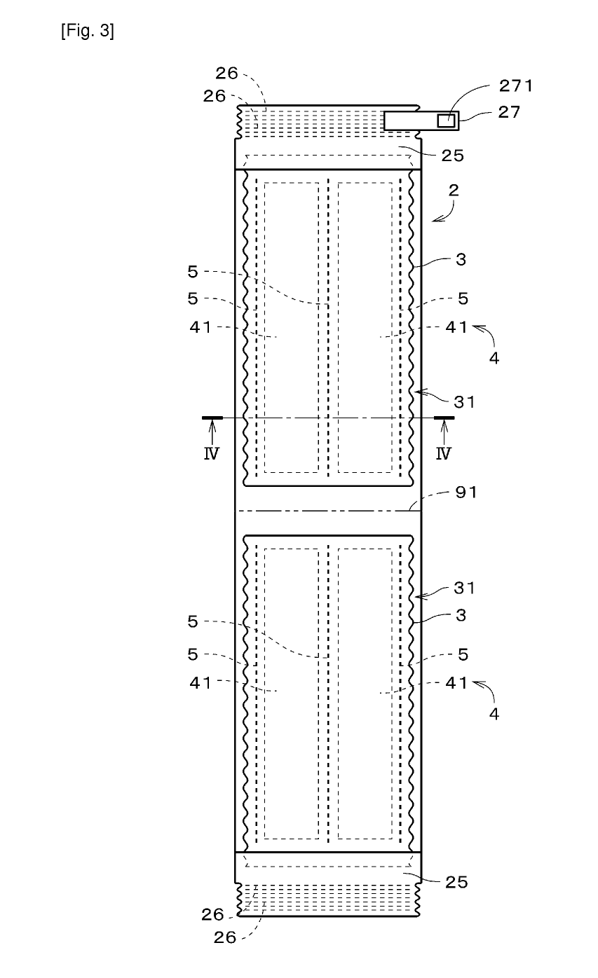

FIG. 3 is a developed view of the absorbent product 1. As shown in FIG. 3, a plurality of elastic members 5 (hereinafter, referred to as “internal sheet elastic members 5”) are bonded to each internal sheet 3 and gathers 31 are formed in the internal sheet 3 by contraction of these internal sheet elastic members 5. In manufacturing of the absorbent product 1, the main body part 2 is folded into two at a folding line 91 extending along a left-right direction orthogonal to the up-down direction at almost the middle in the up-down direction, and with respect to each side edge, side edge parts of both portions sandwiching the folding line 91 (i.e., portions of the main body part 2 which face each other) are bonded to each other by heat sealing or the like.

As shown in FIG. 2, the main body part 2 comprises a bag part 23 having the above-mentioned opening 20 at an upper end into which the male genitalia of the wearer is inserted, absorbent parts 22 attached on an inner surface 231 of the bag part 23 with hot melt adhesive or the like, a liquid-pervious top sheet 21 which covers the inner surface 231 of the bag part 23 with each absorbent part 22 therebetween, liquid-pervious intermediate sheets 24 each of which is located between the absorbent part 22 and the top sheet 21 and covers the absorbent part 22, and opening part sheets 25 bonded to the bag part 23 in the vicinity of the opening 20.

The bag part 23 is a member which is formed of a water-repellent or liquid-impervious sheet member and which is located at the outermost of the absorbent product 1 to prevent urine from leaking out from the inside of the absorbent product 1, and it functions as a so-called back sheet. The upper end part of the bag part 23 is folded inside to overlap with another portion of the bag part 23 and as shown in FIGS. 2 and 3, a plurality of elastic members 26 extending in the left-right direction (hereinafter, the members are referred to as “opening part elastic members 26”) are bonded between above overlapping portions of the bag part 23. As shown in FIG. 3, a fastening part 27 is bonded to one opening part sheet 25 and a field fastener 271 of the fastening part 27 is fastened on an outer surface 232 of the bag part 23 as shown in FIG. 1 so that the size of the opening 20 is adjusted to fit the male genitalia.

As shown in FIG. 2, the top sheet 21 covers almost the entire inner surface 231 of the bag part 23 excluding a part in the vicinity of the opening 20 and it is bonded to the bag part 23 at both side edge parts in the left-right direction of FIG. 3. As shown in FIG. 2, out of the inner surface 231 of the bag part 23, the above part in the vicinity of the opening 20 (i.e., a portion which is not covered with the top sheet 21) is covered with the opening part sheets 25 and the opening part sheets 25 are bonded to the bag part 23. Upper end parts of the top sheet 21 overlap with lower end parts of the opening part sheets 25, and the above-mentioned upper end part of each internal sheet 3 is located between one upper end part of the top sheet 21 and a lower end part of one opening part sheet 25 and is bonded (i.e., fixed) to the top sheet 21 and the opening part sheet 25 at the upper part of the bag part 23. In the absorbent product 1, each absorbent part 22 is located between one internal sheet 3 located in the bag part 23 and the inner surface 231 of the bag part 23, and the top sheet 21 and one intermediate sheet 24 are located between the absorbent part 22 and the internal sheet 3.

The top sheet 21 is a liquid-pervious nonwoven fabric formed from hydrophilic fiber (for instance, cellulose, rayon or cotton) to move urine evacuated from the wearer to the absorbent part 22. For example, a point-bond nonwoven fabric, air-through nonwoven fabric or spunlace nonwoven fabric is utilized as the top sheet 21. A liquid-pervious nonwoven fabric formed from hydrophobic fiber (for instance, polypropylene, polyethylene, polyester, polyamide or nylon) where hydrophilic treatment is performed on its surface with a surfactant, or a plastic film having holes may be utilized as the top sheet 21.

The absorbent part 22 is an absorbent layer which is formed by bonding granular superabsorbent polymer (SAP (Super Absorbent Polymer)) on the inner surface 231 of the bag part 23 with hot melt adhesive or the like and it rapidly absorbs and retains urine from the wearer which has passed through the top sheet 21. The absorbent part 22 may be formed by bonding superabsorbent fiber (SAF (Super Absorbent Fiber)) on the inner surface 231 of the bag part 23 with hot melt adhesive or the like. The absorbent part 22 may be formed, for example, by wrapping a mixture of hydrophilic fiber (crushed pulp fiber, cellulose fiber or the like) and the above-mentioned super-absorbent polymer or superabsorbent fiber in a covering sheet made of a tissue paper, a liquid-pervious nonwoven fabric or the like.

The bag part 23 is a water-repellent or liquid-impervious nonwoven fabric formed from hydrophobic fiber (for instance, polypropylene, polyethylene, polyester, polyamide or nylon) and it prevents urine which has passed through the top sheet 21 from seeping out of the main body part 2. For instance, a spunbond nonwoven fabric, meltblown nonwoven fabric or SMS (spunbond-meltblown-spunbond) nonwoven fabric is utilized as the bag part 23. A water-repellent or liquid-impervious plastic film, or a laminated sheet where the above-mentioned nonwoven fabric is laminated on the outer surface of the plastic film may be utilized for the bag part 23. In the case where the plastic film is utilized for the bag part 23, it is preferred that the plastic film has permeability (i.e., breathability). This can prevent the inside of the absorbent product 1 from becoming stuffy, to increase comfort level of the wearer.

For example, a liquid-pervious nonwoven fabric or a tissue paper is used for the intermediate sheet 24 and urine which has passed through the top sheet 21 is diffused by the intermediate sheet 24. A liquid-pervious sheet member (for instance, a liquid-pervious nonwoven fabric formed from hydrophilic fiber) which is similar to the top sheet 21 is utilized as the opening part sheet 25. String-like members or Band-like members made of polyurethane or natural rubber are used for the opening part elastic members 26. In the preferred embodiment, polyurethane yarns which are elastic yarns are utilized as the opening part elastic members 26.

FIG. 4 is a cross-sectional view of the internal sheet 3 at the position indicated by arrows IV-IV in FIG. 3. As shown in FIGS. 2 and 4, the internal sheet 3 is formed by laminating and bonding two hydrophilic sheets 32 (for instance, two hydrophilic nonwoven fabric formed from hydrophilic fiber such as cellulose, rayon or cotton), and in the preferred embodiment, the internal sheet absorbent part 4 is fixed between the two hydrophilic sheets 32 of the internal sheet 3. The internal sheet absorbent part 4 is an absorbent layer formed from granular superabsorbent polymer (or superabsorbent fiber) bonded with hot melt adhesive or the like between the two hydrophilic sheets 32 of the internal sheet 3 and it rapidly absorbs and retains urine from the wearer.

The internal sheet absorbent part 4 may be formed by wrapping a mixture of hydrophilic fiber (crushed pulp fiber, cellulose fiber or the like) and superabsorbent polymer or superabsorbent fiber in a covering sheet made of a tissue paper, a liquid-pervious nonwoven fabric or the like in the same way as the absorbent part 22 of the main body part 2. The internal sheet 3 may be formed, for example, by folding one hydrophilic sheet into two.

As shown in FIGS. 3 and 4, the internal sheet absorbent part 4 fixed in each internal sheet 3 comprises a plurality of absorbent elements 41 (in the present embodiment, two absorbent elements 41) which extend in the up-down direction and which are arranged in the left-right direction, and the two hydrophilic sheets 32 (see FIG. 4) of the internal sheet 3 are directly bonded to each other at an area between the plurality of absorbent elements 41 (i.e., a nonexistence area of absorber on which superabsorbent polymer is not provided).

The internal sheet elastic members 5 are string-like members or band-like members extending in the up-down direction (i.e., they are almost parallel with the up-down direction) and in the preferred embodiment, three internal sheet elastic members 5 are bonded to each internal sheet 3. Out of the three internal sheet elastic members 5, one internal sheet elastic member 5 is located between the plurality of absorbent elements 41 and is bonded to the two hydrophilic sheets 32 of the internal sheet 3. The other two internal sheet elastic members 5 are bonded to the two hydrophilic sheets 32 at both outer sides (left side and right side) of the internal sheet absorbent part 4 (i.e., two absorbent elements 41). The internal sheet elastic members 5 are made of polyurethane or natural rubber in the same way as the opening part elastic members 26 (see FIG. 3) and in the preferred embodiment, polyurethane yarns which are elastic yarns are utilized as the internal sheet elastic members 5.

The three internal sheet elastic members 5 are bonded to the two hydrophilic sheets 32 in the state where each of them is stretched to a length almost equal to the entire length of the internal sheet 3 in the up-down direction, and by contraction of the internal sheet elastic members 5, gathers 31 are formed in the internal sheet 3 across almost the entire length in the up-down direction as shown in FIGS. 2 and 3.

When the absorbent product 1 is worn, the male genitalia is inserted into the inside of the main body part 2 through the opening 20 shown in FIG. 2 and it is located between the two internal sheets 3. Then, the fastening part 27 (see FIG. 1) is fastened on the outer surface 232 of the bag part 23 so that the size of the opening 20 is adjusted to fit the male genitalia. When urine is excreted in the inside of the absorbent product 1, the urine is received by the hydrophilic internal sheets 3, and the urine is led to the bottom part of the main body part 2 (i.e., the bottom part which is on the opposite side to the opening 20 of the bag part 23) while it penetrates into the internal sheets 3. Some of urine which penetrates into the internal sheet 3 is absorbed by the internal sheet absorbent part 4, and urine which has passed through the internal sheet 3 without absorption by the internal sheet absorbent part 4 passes through the top sheet 21 and the intermediate sheet 24 of the main body part 2 contacting the internal sheet 3, to be absorbed by the absorbent part 22.

In the absorbent product 1, since urine moves in the internal sheet 3 comparatively slowly toward the bottom part of the bag part 23 with passing through the gathers 31 formed in the internal sheet 3, a time (time period) required for the urine to reach the bottom part of the bag part 23 can be increased. As the result, while the urine spreads over a large area on the liquid-pervious internal sheet 3, it moves to the main body part 2 through the internal sheet 3, and then it is absorbed over a large area of the absorbent part 22. As above, since a large area of the absorbent part 22 can be utilized to absorb urine effectively in the absorbent product 1, it is prevented that urine stays in the inside of the bag part 23 without being absorbed. It is therefore possible to prevent that some of urine which is not absorbed flows back from the inside of the absorbent product 1 to leak out through a gap between the opening 20 and the male genitalia.

As described above, since the internal sheet 3 is the hydrophilic sheet member, urine easily penetrates into the internal sheet 3, and therefore a moving speed where urine received by the internal sheet 3 moves to the bottom part of the bag part 23 decreases further. As the result, a time required for the urine to reach the bottom part of the bag part 23 can be increased further and a larger area of the absorbent part 22 can be utilized to absorb the urine more effectively.

Since the absorbent product 1 comprises the two internal sheets 3 in the bag part 23 which face each other, a time required for urine to reach the bottom part of the bag part 23 can be increased still further and large areas of the absorbent parts 22 can be utilized to absorb the urine still more effectively. The gathers 31 are formed from the upper part to the lower part of each internal sheet 3 (they need not be formed across the entire length of the internal sheet 3 in the up-down direction). Therefore, a time required for urine received by the internal sheet 3 to reach the bottom part of the bag part 23 can be increased still further to absorb the urine still more effectively.

As described above, the upper end part of the internal sheet 3 is fixed on the upper part of the main body part 2 (i.e., the part located in the vicinity of the opening 20) and portions of the internal sheet 3 excluding the upper end part are not bonded to (the top sheet 21 of) the main body part 2. Thus, gathers by contraction of the internal sheet elastic members 5 are not formed in the main body part 2, and this can prevent the absorbent product 1 from becoming bulky (thick) and make it easy to handle the absorbent product 1. Since the internal sheet elastic members 5 are string-like members or band-like members extending in the up-down direction, the gathers 31 formed by contraction of the internal sheet elastic members 5 have construction where a plurality of creases extend almost in the left-right direction. Therefore, a time required for urine to reach the bottom part of the bag part 23 across the gathers 31 can be increased further and as the result, a large area of the absorbent part 22 can be utilized to absorb the urine more effectively.

Since the absorbent product 1 is provided with the internal sheet absorbent part 4 attached to the internal sheet 3, absorbency of the absorbent product 1 as a whole can be increased. As the result, it is possible to reliably prevent urine from leaking out from the absorbent product 1. The internal sheet absorbent part 4 is formed from superabsorbent polymer or superabsorbent fiber (in other words, any bulky member such as crushed pulp, is not included in the internal sheet absorbent part 4 and the internal sheet absorbent part 4 is formed by using only superabsorbent polymer or superabsorbent fiber as an absorber). Thus, it is achieved to reduce a thickness of the internal sheet 3 and increase softness of the internal sheet 3, and therefore it is possible to easily form the gathers 31 and prevent the absorbent product 1 from becoming bulky. Furthermore, the absorbent part 22 of the main body part 2 is formed from superabsorbent polymer or superabsorbent fiber, and this further prevents the absorbent product 1 from becoming bulky.

As described above, the internal sheet absorbent part 4 comprises the plurality of absorbent elements 41 which each extends in the up-down direction and which are arranged in the left-right direction, and an area where superabsorbent polymer does not exist is provided between the plurality of absorbent elements 41. Therefore, when the internal sheet absorbent part 4 absorbs urine, it is prevented that swelling of each absorbent element 41 from being inhibited by the other absorbent element 41, so that absorbency of the internal sheet absorbent part 4 can be increased. Even though the absorbent elements 41 have fully swollen, urine passes through each area between the plurality of absorbent elements 41 to be reliably led to the bottom part of the bag part 23.

The internal sheet elastic members 5 are bonded to the two hydrophilic sheets 32 of the internal sheet 3 at the area (portion) between the plurality of absorbent elements 41 and the areas lying at both outer sides of the internal sheet absorbent part 4 where superabsorbent polymer does not exist. Therefore, the internal sheet elastic members 5 can be bonded to the internal sheet 3 firmly, and it is possible to prevent the internal sheet elastic members 5 from dropping from the internal sheet 3 after the internal sheet absorbent part 4 has fully swollen. The areas on the internal sheet 3 where superabsorbent polymer does not exist are softer than areas where superabsorbent polymer exists. Thus, the gathers 31 can easily be formed in the internal sheet 3 by allocating the internal sheet elastic members 5 on such the soft areas.

FIGS. 5 to 7 are views each showing another shape example of internal sheet absorbent part. In FIGS. 5 to 7, the internal sheet 3 and the internal sheet elastic members 5 are drawn and hatching lines are drawn at absorbent elements 41 of the internal sheet absorbent part 4a, 4b, 4c for easy understanding of the drawing. In the internal sheet absorbent part 4a shown in FIG. 5, three absorbent elements 41 are arranged in the up-down direction and two nonexistence areas 42 of absorber (i.e., the areas to which superabsorbent polymer is not bonded) lying across the internal sheet absorbent part 4a in the left-right direction are provided. Therefore, stiffness of the internal sheet 31 in the up-down direction is decreased and the gathers 31 are easily formed in the internal sheet 3.

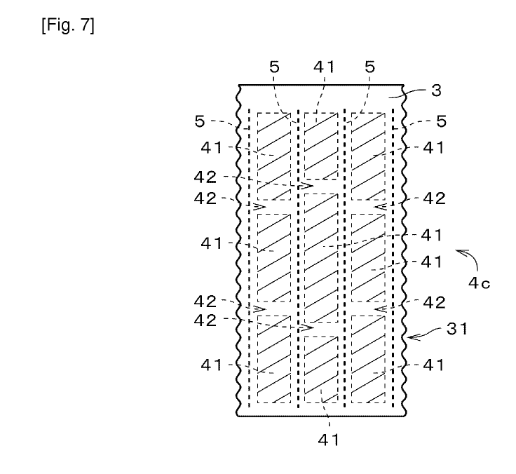

In the internal sheet absorbent part 4b shown in FIG. 6, four absorbent elements 41 are arranged in a lattice pattern and one nonexistence area 42 of absorber lying across the internal sheet absorbent part 4b in the left-right direction is provided. In the internal sheet absorbent part 4c shown in FIG. 7, three absorbent element lines each of which comprises three absorbent elements 41 aligned in the up-down direction are arranged in the left-right direction. In the internal sheet absorbent part 4c, two nonexistence areas 42 of absorber are provided in each absorbent element line, and with respect to the up-down direction, positions of the nonexistence areas 42 in the central absorbent element line are different from positions of the nonexistence areas 42 in the other two absorbent element lines. In other words, in the internal sheet absorbent part 4c, two nonexistence areas 42 of absorber each of which extends in left-right direction in a zigzag manner, lie across the internal sheet absorbent part 4c in the left-right direction. In the internal sheet absorbent parts 4b, 4c, the gathers 31 are easily formed in the internal sheet 3 in a similar fashion to the internal sheet absorbent part 4a (see FIG. 5).

Though the preferred embodiments of the present invention have been discussed above, the present invention is not limited to the above-discussed preferred embodiments, but allows various variations.

The gathers 31 are not necessarily formed in the internal sheet 3 across almost the entire length in the up-down direction, and the gathers 31 have only to be formed in at least part of the internal sheet 3. For example, the gathers 31 may be formed at a portion of the internal sheet 3 opposed to a lower part of the bag part 23. In the absorbent product 1, by forming the gathers 31 at the portion of the internal sheet 3 opposed to the lower part of the bag part 23, a time required for urine received by the internal sheet 3 to reach the bottom part of the bag part 23 can be increased to absorb the urine effectively.

Though the internal sheet absorbent part comprising the plurality of absorbent elements 41 has been discussed in the above preferred embodiments, the internal sheet absorbent part may comprise only one absorbent element 41. In the case where the absorbent part 22 of the main body part 2 has enough absorbency, the internal sheet absorbent part may be omitted.

In the above preferred embodiments, the sheet member to form the bag part 23 is folded into two at the folding line 91 along the left-right direction in FIG. 3 and both side edge parts are bonded. However, the bag part 23 may be formed by folding one sheet member into two at a folding line along the up-down direction and bonding both portions sandwiching the folding line to each other at the lower edge part and one side edge part. The internal sheet 3 may be formed similarly by folding one sheet member into two at a folding line along the up-down direction. In this case, the male genitalia is located between both portions sandwiching the folding line.

In the absorbent product 1, two internal sheets 3 are provided in the main body part 2. However, as long as a time required for urine to reach the bottom part of the bag part 23 can be increased enough, one internal sheet 3 may be omitted. Though the internal sheet 3 and the top sheet 21 are individual members, the internal sheet 3 and the top sheet 21 may be formed by one long hydrophilic sheet member. In this case, the hydrophilic sheet member is folded into two at the upper part of the bag part 23 and one of portions sandwiching the folding line is bonded to the inner surface 231 of the bag part 23 to function as the top sheet 21. The other of the portions sandwiching the folding line is located in the inside of the bag part 23 to function as the internal sheet 3.

In the main body part 2, a loop member on which the field fastener 271 of the fastening part 27 is fastened may be provided on the outer surface 232 of the bag part 23. An adhesive tape may be provided in the fastening part 27 as substitute for the field fastener 271. In this case, a target tape on which the adhesive tape is fastened may be provided on the outer surface 232 of the bag part 23.

In the absorbent product 1, there may be a case where the top sheet 21 is omitted and the absorbent part 22 located on the inner surface 231 of the bag part 23 is covered with an internal sheet 3 which is bonded to the inner surface 231 of the bag part 23 or an internal sheet 3 which is not bonded to the inner surface 231 of the bag part 23. In this case, it is preferred that the internal sheet 3 is bonded to the inner surface 231 of the bag part 23 in order to reliably fix the absorbent part 22. The top sheet 21 and the absorbent part 22 may be omitted and in this case, urine is absorbed by the internal sheet absorbent part 4.

While the invention has been shown and described in detail, the foregoing description is in all aspects illustrative and not restrictive. It is therefore understood that numerous modifications and variations can be devised without departing from the scope of the invention.

### REFERENCE SIGNS LIST

1 absorbent product
3 internal sheet
4, 4a to 4c internal sheet absorbent part
5 internal sheet elastic member
20 opening
21 top sheet
22 absorbent part
23 bag part
31 gathers
41 absorbent element
42 nonexistence area of absorber
231 inner surface

1. An absorbent product (1) for receiving urine from a male wearer, comprising:
a bag part (23) which is formed of a water-repellent or liquid-impervious sheet member and which has an opening (20) at an upper end into which the male genitalia of a wearer is inserted;
a liquid-pervious internal sheet (3) located in said bag part;
an absorbent part (22) for absorbing urine from said wearer, said absorbent part being located between said bag part and said internal sheet; and
an elastic member (5) which is bonded to said internal sheet, gathers (31) being formed in at least part of said internal sheet by contraction of said elastic member.

2. The absorbent product according to claim 1, wherein said gathers are formed at a portion of said internal sheet opposed to a lower part of said bag part.

3. The absorbent product according to claim 1, wherein
said gathers are formed from an upper part to a lower part of said internal sheet.

4. The absorbent product according to claim 1, wherein said absorbent part is formed from superabsorbent polymer or superabsorbent fiber.

5. The absorbent product according to claim 1, further comprising
a liquid-pervious top sheet (21) which is located between said absorbent part and said internal sheet and which covers an inner surface (231) of said bag part, wherein
said internal sheet is a band-like member whose upper end part is fixed on an upper part of said bag part.

6. The absorbent product according to claim 5, wherein
said elastic member is a string-like member or a band-like member extending in an up-down direction.

7. The absorbent product according to claim 5, further comprising an internal sheet absorbent part (4, 4a to 4c) which is attached to said internal sheet.

8. The absorbent product according to claim 7, wherein
said internal sheet absorbent part is formed from superabsorbent polymer or superabsorbent fiber.

9. The absorbent product according to claim 7, wherein
said internal sheet absorbent part comprises a plurality of absorbent elements (41) which extend in an up-down direction and which are arranged in a direction orthogonal to said up-down direction, and
said elastic member is a string-like member or a band-like member extending in said up-down direction and said elastic member is bonded to said internal sheet at an area between said plurality of absorbent elements.

10. The absorbent product according to claim 7, wherein
a nonexistence area (42) of absorber lying across said internal sheet absorbent part in a direction orthogonal to an up-down direction is provided on said internal sheet absorbent part.

11. The absorbent product according to claim 5, further comprising another internal sheet (3) whose upper end part is fixed on an upper part of said bag part and which faces said internal sheet.

12. The absorbent product according to claim 2, further comprising
a liquid-pervious top sheet (21) which is located between said absorbent part and said internal sheet and which covers an inner surface (231) of said bag part, wherein
said internal sheet is a band-like member whose upper end part is fixed on an upper part of said bag part.

13. The absorbent product according to claim 3, further comprising
a liquid-pervious top sheet (21) which is located between said absorbent part and said internal sheet and which covers an inner surface (231) of said bag part, wherein
said internal sheet is a band-like member whose upper end part is fixed on an upper part of said bag part.

14. The absorbent product according to claim 4, further comprising
a liquid-pervious top sheet (21) which is located between said absorbent part and said internal sheet and which covers an inner surface (231) of said bag part, wherein
said internal sheet is a band-like member whose upper end part is fixed on an upper part of said bag part.
